(19) United States
(12) Patent Application Publication
(10) Pub. No.: US 2025/0114375 A1
(43) Pub. Date: Apr. 10, 2025

(54) PHARMACEUTICAL COMPOSITION OF LIFITEGRAST AND LOTEPREDNOL ETABONATE

(71) Applicant: ORBICULAR PHARMACEUTICAL TECHNOLOGIES PRIVATE LIMITED, Hyderabad (IN)

(72) Inventor: Hiren PATEL, Hyderabad (IN)

(21) Appl. No.: 18/836,057

(22) PCT Filed: Feb. 8, 2023
(86) PCT No.: PCT/IB2023/051101

§ 371 Date: Aug. 6, 2024

(30) Foreign Application Priority Data
Feb. 8, 2022 (IN) 202241006646

(51) Int. Cl. A61K 31/567; A61K 31/4725

(52) U.S. Cl. CPC A61K 31/567; A61K 31/4725

(57) ABSTRACT

The present invention relates to pharmaceutical composition comprising combination of Lifitegrast and Loteprednol etabonate along with one or more pharmaceutically acceptable excipients for the treatment of dry eye disease.

### FIELD OF THE INVENTION

This invention relates to pharmaceutical composition comprising combination of Lifitegrast and Loteprednol etabonate along with one or more pharmaceutically acceptable excipients.

### BACKGROUND OF THE INVENTION

Dry Eye Disease (DED) is a common ocular surface disorder affecting millions of people worldwide. DED is also called keratoconjunctivitis sicca (KCS), sicca syndrome, keratitis sicca, xerophthalmia, dry eye syndrome (DES), dysfunctional tear syndrome (DTS), ocular surface disease (OSD) or dry eye.

There is currently no single globally accepted definition of DED available. As per the Dry Eye Workshops (DEWS™) I and II of the Tear Film & Ocular Surface Society (TFOS) dry eye is defined as a multifactorial disease of the ocular surface characterized by a loss of homeostasis of the tear film, and accompanied by ocular symptoms, in which tear film instability and hyperosmolarity, ocular surface inflammation and damage, and neurosensory abnormalities play etiological roles. DED is a serious chronic or progressive disease rather than a temporary syndrome.

Dry eye disease is a disorder of the tear film that are due to reduced tear production or tear film instability, associated with ocular discomfort and/or visual symptoms and inflammatory disease of the ocular surface. Hence inflammation is considered to play an important role in the dry eye disease.

DED is typically categorized into two general subtypes such as the aqueous deficient-type and the evaporative-type of DED. Aqueous deficient DED is characterized by a reduced tear production, due to e.g., dysfunctional lacrimal glands, which then leads to a reduced aqueous tear or increased osmolarity of the tear film and finally to an insufficiently protected ocular surface. Evaporative DED is characterized by rather normal aqueous tear production, but pathologically increased evaporation of the tear fluid. The lipid part of the tear film provides a natural barrier for excessive tear evaporation. A reduced or porous lipid layer does not sufficiently prevent aqueous tear evaporation. Evaporative DED is often associated with a Meibomian Gland Dysfunction (MGD) as secretion from the Meibomian glands is the sole natural source of the lipid layer.

Both types of DED result in tear film instability and consequently in ocular surface damage as the eye surface is not adequately protected against blinking and environmental stress. The clinical experience and respective treatment guidelines also suggest that DED patients cannot be adequately treated with one universal therapy but require specific treatment for their individual type of DED and the resulting condition. The dry eye disease, if left untreated and uncorrected, can result in permanent damage to the eye with degradation of the exposed ocular tissues or a breakdown of the corneal tissue necessitating, in extreme cases, corneal transplants.

Currently two treatment options are available such as artificial tears or lubricants and prescription drugs. Artificial tears treat subjective symptoms of DED but not clinical signs of DED while prescription drugs are thought to treat both signs and symptoms of DED.

Currently only four drugs are FDA approved for the treatment of dry eye disease. Cyclosporine is approved as 0.05% ophthalmic emulsion to Allergan under the tradename Restasis® and Restasis Multidose® and 0.09% micellar solution to Sun pharma under the tradename Cequa®, Lifitegrast (Xiidra®) 5% ophthalmic solution to Novartis, Loteprednol etabonate (Eysuvis®) 0.25% ophthalmic suspension of Kala Pharmaceuticals and Varenicline (Tyrvaya®) 0.03 mg nasal spray solution of Oyster Point Pharma. The compounded formulations of Cyclosporine 0.1% ophthalmic emulsion PF (Klarity-C Drops®) and in combination with loteprednol 0.2% (Klarity-CL Drops suspension), Glycerin 1% ophthalmic solution PF (Klarity Drops®) are also available in US. The other two mucin secretagogues Diquafosol (Diquas®) 3% ophthalmic solution for topical use as eye drop by Santen and Rebamipide (Mucosta®) 2% ophthalmic suspension for topical use as eye drop by Otsuka Pharmaceuticals are commercially available in Japan and few Asian markets, which are intended to promote mucin production and are expected to increase tear film stability.

Cyclosporine is a calcineurin inhibitor immunosuppressant with anti-inflammatory effects. Restasis® and Restasis Multidose® are 0.05% ophthalmic emulsion of cyclosporine which are administered topically as one drop twice a day in each eye approximately 12 hours apart to increase tear production in patients whose tear production is presumed to be suppressed due to ocular inflammation associated with keratoconjunctivitis sicca. CEQUA® is a 0.09% ophthalmic solution of cyclosporine which is administered topically one drop, twice daily (approximately 12 hours apart) into each eye to increase tear production in patients with keratoconjunctivitis sicca (dry eye).

Lifitegrast (Xiidra®) is a lymphocyte function-associated antigen-1 (LFA-1) antagonist approved as 5% ophthalmic solution which is administered topically as one drop twice daily (approximately 12 hours apart) into each eye using a single-use container for the treatment of the signs and symptoms of dry eye disease (DED). Lifitegrast binds to the integrin LFA-1, a cell surface protein found on leukocytes and blocks the interaction of LFA-1 with its cognate ligand intercellular adhesion molecule-1 (ICAM-1). ICAM-1 may be overexpressed in corneal and conjunctival tissues in DED. LFA-1/ICAM-1 interaction can contribute to the formation of an immunological synapse resulting in T-cell activation and migration to target tissues. In vitro studies demonstrated that lifitegrast may inhibit T-cell adhesion to ICAM-1 in a human T-cell line and may inhibit secretion of inflammatory cytokines in human peripheral blood mononuclear cells.

Loteprednol (Eysuvis®) is a corticosteroid is approved as 0.25%, ophthalmic suspension which is topically administered as one to two drops of into each eye four times daily for up to two weeks for the short-term (up to two weeks) treatment of the signs and symptoms of dry eye disease. Corticosteroids inhibit the inflammatory response to a variety of inciting agents and delay or slow healing. Corticosteroids inhibit the edema, fibrin deposition, capillary dilation, leukocyte migration, capillary proliferation, fibroblast proliferation, deposition of collagen, and scar formation associated with inflammation. While glucocorticoids are known to bind to and activate the glucocorticoid receptor, the molecular mechanisms involved in glucocorticoid/glucocorticoid receptor-dependent modulation of inflammation are not clearly established. However, corticosteroids are thought to inhibit prostaglandin production. There are other approved corticosteroids which are used to treat the general ocular inflammation not specifically associated with DED.

The U.S. Pat. No. 10,555,947 discloses ophthalmic composition comprising: a first ophthalmic compound and a second ophthalmic compound that is different from said first ophthalmic compound, wherein said first ophthalmic compound is selected from the group consisting of an alpha 2 adrenergic agonist and a corticosteroid, and wherein said second ophthalmic compound is selected from the group consisting of: (i) an alpha 2 adrenergic agonist; (ii) a corticosteroid (iii) a lymphocyte function-associated antigen antagonist; (iv) a sodium channel blocker; (v) a non-steroidal anti-inflammatory drug; (vi) an antibiotic; and (vii) a combination of two or more thereof. U.S. Pat. No. 10,555,947 specifically teaches the first ophthalmic compound is brimonidine and second ophthalmic agent is cyclosporine.

The U.S. Pat. No. 8,084,047 claims Lifitegrast drug substance and generically discloses LFA-1 antagonist may be administered in combination with other active agents includes corticosteroids as one of the agent from the long list.

The other US patents and patent applications U.S. Pat. No. 8,367,701, US20090258070A1, US20140051722A1 specifically related to composition comprising an LFA-1 antagonist (Lifitegrast) and generically discloses that the composition comprises additional one or more therapeutically active agents including corticosteroids as one of the example from long list.

The PCT application WO2020/072971A1 discloses stable aqueous topical ophthalmic composition comprising about 0.1% to 10% of cevimeline at a neutral pH range of pH 6-9, and one or more pharmaceutically acceptable excipients. Further generically discloses that composition comprises polyunsaturated fatty acids (PUFA), one or more other active ingredient(s) selected from the group consisting of a) at least one steroid; b) at least one immunomodulator (immunosuppressant); c) at least one hormone (androgen); d) a lymphocyte function associated antigen-1 (LFA-1) antagonist; and f) at least one active ingredient effective for treating Meibomian gland dysfunction.

The U.S. Pat. No. 10,688,045 discloses to topical pharmaceutical composition comprising loteprednol, mucus penetration-enhancing agents, glycerin, surface active agents, wherein the surface active agents includes span 20, span 80, or octyl glucoside (triton×100). Further, it teaches when nanosuspensions comprises surface-altering agents such as Pluronics® L101, L81, L44, L31, span 20, span 80, or octyl glucoside (triton X100), stable nanosuspensions could not be obtained due to their inability to effectively aid particle size reduction.

The U.S. Pat. No. 8,835,410 related to method for the treatment of eyelid dermatitis and/or dermatitis of the periorbital area applying composition comprises loteprednol etabonate and A1-cortienic acid, wherein composition in the form of ointment, a cream, a gel, a cream gel or a lotion. Further, exemplified aqueous gel composition of loteprednol etabonate and Δ1-cortienic acid with xanthan gum, other excipients. Specifically teaches combination of steroid drugs for eyelid dermatitis and exemplified aqueous gel composition with xanthan gum.

The US patent publication 20120053161A1 disclosed ophthalmic, otic or nasal composition consisting of water-insoluble corticosteroid, cyclodextrin, xanthan gum, water, wherein the corticosteroid includes dexamethasone, fluorometholone, prednisolone, loteprednol, rimexolone. Further, exemplified composition comprises dexamethasone with xanthan gum, other excipients. US20120053161 discloses ophthalmic, otic or nasal composition, further disclosed broadly loteprednol is one of active agent and cyclodextrin, xanthan gum.

None of the above patents or patent applications teaches or discloses the combination of Lifitegrast with Loteprednol etabonate.

Control of inflammation is considered mandatory in order to improve symptomatology, direct inflammatory damage or lack of epithelial protection can make free nerve endings of corneal epithelium more sensitive to normal and environmental stimuli inducing neuropathic pain, a typical symptom of DED. The control and reduction of ocular surface inflammation which may derive from epithelial damage and environmental stressors, is another key component of any treatment regimen (Aragona P et al., Towards a dynamic customised therapy for ocular surface dysfunctions. Br J Ophthalmol, 2013).

The current treatment options are limited and leave patients and ophthalmologists unsatisfied, resulting in a high unmet medical need. Additionally, increasing prevalence and several risk factors like aging population and screen work, make innovation and new treatment options necessary.

The commercially available products and products known in the art used alone for treatment of dry eye. The DED requires chronic treatment and persistent effects to control the inflammation. Monotherapy treatment options are limited and do not provide satisfactory improvement of signs and symptoms of dry eye disease.

Initial phase of DED symptoms are critical to manage, as it will have inflammation and severe symptoms. Hence to combat this the present has tried to provide necessary treatment of the combination composition to reduce the signs and symptoms of DED.

Thus, there is an unmet need in the art to provide composition which will provide improved efficacy and better patient compliance and to relieve the signs and symptoms of dry eye disease. Hence, the present invention provides pharmaceutical composition comprising combination of two drugs which provides synergistic effect for reducing ocular surface inflammation associated with dry eye disease. The present invention provides pharmaceutical composition comprising combination of Lifitegrast and Loteprednol etabonate which synergistically provides improvement in signs and symptoms of dry eye disease.

### SUMMARY OF THE INVENTION

One object of the present invention is to provide a pharmaceutical composition comprising combination of Lifitegrast or pharmaceutically acceptable salts thereof and Loteprednol or pharmaceutically acceptable salts thereof.

One object of the present invention is to provide a pharmaceutical composition comprising combination of Lifitegrast and Loteprednol etabonate.

Another object of the present invention is to provide a pharmaceutical composition comprising combination of Lifitegrast and Loteprednol or pharmaceutically acceptable salts in the form of solution, suspension, emulsion, gel, gel forming solution, dispersion, nano-formulations, insert, implant, punctal plugs and the like.

Another object of the present invention is to provide a pharmaceutical composition comprising combination of Lifitegrast and Loteprednol or pharmaceutically acceptable salts thereof along with one or more pharmaceutically acceptable excipient.

Another object of the present invention, the pharmaceutical composition of Lifitegrast and Loteprednol comprises one or more pharmaceutically acceptable excipient, wherein the excipients include buffering agents, pH-adjusting agents, preservatives, osmotic/tonicity adjusting agents, viscosity modifying agents, antioxidants, surfactants or surface active agents, stabilizing agents, chelating agents, vehicle and the like.

Another object of the present invention, the pharmaceutical composition of Lifitegrast and Loteprednol comprises buffering agents include sodium citrate dihydrate, sodium phosphate dibasic anhydrous, citric acid monohydrate; pH-adjusting agent include sodium hydroxide; preservatives include benzalkonium chloride; osmotic/tonicity adjusting agent include sodium chloride, gycerin; viscosity modifying agents include xanthan gum, guar gum; surfactants or surface active agents include polyoxyethylene (40) octyl phenyl ether; chelating agent include edetate disodium dihydrate; vehicle include water for injection and combinations thereof.

Another object of the present invention is to provide a pharmaceutical composition comprising combination of Lifitegrast and Loteprednol or pharmaceutically acceptable salts thereof for the treatment of dry eye disease.

### DETAILED DESCRIPTION OF THE INVENTION

In one aspect, present invention provides pharmaceutical composition comprising: Lifitegrast or pharmaceutically acceptable salts thereof and Loteprednol or pharmaceutically acceptable salts thereof.

In another aspect, present invention provides pharmaceutical composition comprising: combination of Lifitegrast or pharmaceutically acceptable salts thereof and Loteprednol or pharmaceutically acceptable salts thereof.

In another aspect, present invention provides pharmaceutical composition comprising: combination of Lifitegrast and Loteprednol etabonate.

In another aspect, present invention provides pharmaceutical composition comprising: combination of Lifitegrast and Loteprednol or pharmaceutically acceptable salts thereof along with one or more pharmaceutically acceptable excipients.

In another aspect, present invention provides pharmaceutical composition comprising: combination of Lifitegrast and Loteprednol or pharmaceutically acceptable salts thereof along with one or more pharmaceutically acceptable excipients for the treatment of dry eye disease.

The term “active pharmaceutical ingredient” or “API” or “drug” refers to a substance that has a physiological effect when ingested or otherwise introduced into the body, in particular or a chemical substance used in the treatment, cure, prevention, or diagnosis of disease or used to otherwise enhance physical or mental well-being.

The term “dry eye disease” or “DED” includes keratoconjunctivitis sicca (KCS), sicca syndrome, keratitis sicca, xerophthalmia, dry eye syndrome (DES), evaporative dry eye (EDE), aqueous tear-deficient dry eye (ADDE), dysfunctional tear syndrome (DTS), ocular surface disease (OSD), dry eye, Meibomian gland disease, Meibomian gland dysfunction, Sjogren's syndrome (also known as “Mikulicz disease” and “Sicca syndrome”), non Sjogren's syndrome dry eye, dry eye syndrome due to a complication of refractive eye surgery.

The term “composition” is intended to encompass a combination including active ingredients and pharmaceutically acceptable excipients, as well as any product which results, directly or indirectly, from combination, complexation, or aggregation of any two or more of the ingredients, or from dissociation of one or more of the ingredients, or from other types of reactions or interactions involving one or more of the ingredients.

The term “formulation” or “dosage form” or “composition” refers to finished pharmaceutical products that are suitable for topical administration to eye, including, but not limited to, ophthalmic dosage forms such as solution, suspension, emulsion, dispersion, ointment, gel or gel forming solution, nano-formulations, micellar solution, nanoemulsion, nanosuspension, insert, implants, punctal plugs, microneedle, contact lenses, intraocular injection etc.

The term “excipient” or “pharmaceutically acceptable excipient” means a physiologically compatible additives useful in preparation of a pharmaceutical composition. The excipients or adjuvants that are useful in preparing pharmaceutical compositions are generally safe, non-toxic, and neither biologically nor otherwise undesirable, and are acceptable for human pharmaceutical use as well as veterinary use. The term includes one or more excipients or adjuvants.

The term “pharmaceutically acceptable salts” includes pharmaceutically acceptable salts, solvates, hydrates, anhydrates, enantiomers, esters, isomers, polymorphs, tautomers, complexes and thereof. The derivatives of the parent compounds wherein the parent compound is modified by making acid or base salts, solvate, hydrate, esters and the like thereof. Pharmaceutically-acceptable salt forms of compounds provided herein are synthesized from the parent compound which contains a basic or acidic moiety by conventional chemical methods. Pharmaceutically acceptable salts of amines, carboxylic acids, and other types of compounds, are well known in the art. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include ethyl carbonate or etabonate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “optional” or “optionally” means that the subsequently described element, component or circumstance may or may not be present, so that the description includes instances where the element, component, or circumstance is included and instances where it is not.

The term “about” when used in conjunction with a numeral here refers to a range of that numeral +/−10%, inclusive. However, alternative concentrations are also expressly deemed suitable for use herein.

The term “combination” refers to the composition comprises single pharmaceutically active agent or mixture or combination of one or more pharmaceutically active agents or composition composed of any combination of a drug and a device, or a drug and a drug, or drug and one or more pharmaceutically excipients. One or more pharmaceutically active agents in a single dosage form or different dosage form in any ratio or concentration and/or fixed ratio or concentration and/or different ratio or concentration.

One or more pharmaceutically active agents can be administered simultaneously or Sequential administration includes administration before or after each other. In some embodiments, the additional therapeutic agent or agents can be administered in the same composition with first active agent. Combination of active agents can be administering in separate composition or in the same composition in any ratio or concentration and/or fixed ratio or concentration and/or different ratio or concentration. In other embodiments, there can be an interval of time between administration of the one or more pharmaceutically active agents.

In one embodiment, the present invention provides combination of Lifitegrast or pharmaceutically acceptable salts thereof and Loteprednol or pharmaceutically acceptable salts.

In one embodiment, the present invention provides combination of Lifitegrast and Loteprednol etabonate.

Lifitegrast is an N-acyl-L-alpha-amino acid obtained by formal condensation of the carboxy group of N-[2-(1-benzofuran-6-carbonyl)]-5,7-dichloro-1,2,3,4-tetrahydroisoquinoline-6-carboxylic acid with the amino group of 3-(methanesulfonyl)-L-phenylalanine. It has a role as an anti-inflammatory drug and a lymphocyte function-associated antigen-1 antagonist. It is a L-phenylalanine derivative, a sulfone, a N-acyl-L-alpha-amino acid, a member of isoquinolines and a member of 1-benzofurans.

It is well understood from the literature that Lifitegrast act by inhibiting lymphocyte migration to the ocular surface but require a longer period compared to corticosteroids before they become effective in controlling inflammation (Perez V L et al, Lifitegrast, a novel integrin antagonist for treatment of dry eye disease. Ocular Surface, 2016). Lifitegrast increase the number of conjunctival goblet cells and goblet cell area by 39% and 22% respectively (Stacy L. Haber et al, Lifitegrast: a novel drug for patients with dry eye disease, Adv Ophthalmol, 2019).

Lifitegrast reduces ocular surface inflammation, with a unique mechanism of action. The potential advantage is that it may work as soon as 2 weeks which is earlier than may be achieved with cyclosporine (Craig J P et al, TFOS DEWS II report executive summary, Ocular Surface, 2017 and Tauber J et al., randomized phase III OPUS-2 study Ophthalmology, 2015).

Corticosteroids are typically used in treatment of ocular surface inflammation. Corticosteroids treatment with high-medium potency molecules has been demonstrated to be effective. Other molecules can be used to treat inflammation alone or in combination with corticosteroids. Other molecules like Cyclosporine and Lifitegrast have been indicated as possible treatment acting on specific aspects of inflammation (Marsh P et al., Topical methylprednisolone therapy, Ophthalmology 1999; Pflugfelder S C et al., Loteprednol etabonate, Am J Ophthalmol, 2004; Aragona P et al., 0.1% Clobetasone butyrate, Eur J Ophthalmol, 2013).

Corticosteroids inhibit the conversion of phospholipids to arachidonic acid by inhibiting the catalytic enzyme phospholipase A2 in the synthetic pathway. Steroids work higher up in the arachidonic acid synthetic pathway, they inhibit cyclooxygenase and lipoxygenase, thus inhibiting production of prostaglandins and leukotrienes which are powerful mediators of inflammation.

Loteprednol etabonate (“LE”) is a known soft corticosteroid based on the known inactive metabolite prednisolone acetate of the active drug prednisolone (U.S. Pat. Nos. 4,996,335 and 4,710,495). Loteprednol etabonate is an etabonate ester, an 11beta-hydroxy steroid, a steroid ester, an organochlorine compound, a steroid acid ester and a 3-oxo-Delta(1), Delta(4)-steroid. It has a role as an anti-inflammatory drug. It derives from a loteprednol. LE is an analog of prednisolone that does not have a 20-keto group attached to the 17β-position. Instead, the 17β-position is occupied with a metabolically-labile ester function. In biological systems, LE is hydrolysed to the inactive carboxylic acid metabolite (PJ-91) that does not bind to glucocorticoid receptors. LE also provides superior safety by reducing the risk of steroid induced cataracts and elevation of intra-ocular pressure. The ability of LE to enzymes located in the blood and/or liver also reduces the likelihood of systemic side effects. LE therefore provides therapeutic advantages over other corticosteroids by providing efficacy similar to its parent compound, namely, prednisolone acetate, with fewer deleterious systemic side effects. Soft steroids have the potential advantage of treating inflammation without inducing elevation of intraocular pressure. In addition, soft steroids can provide the added benefit of a lower tendency to induce cataracts which may result from interaction of corticosteroids with the ocular lens proteins.

In one embodiment, the present invention provides pharmaceutical composition comprising: a) Lifitegrast or pharmaceutically acceptable salts thereof; b) Loteprednol or pharmaceutically acceptable salts thereof and c) one or more pharmaceutically acceptable excipients.

In one embodiment, the present invention provides pharmaceutical composition comprising: a) Lifitegrast; b) Loteprednol etabonate and c) one or more pharmaceutically acceptable excipients.

The pharmaceutical composition of the present invention comprises Lifitegrast or pharmaceutically acceptable salts thereof in the concentration range from about 0.01% w/v to about 20% w/v; from about 0.1% w/v to about 10% w/v; from about 1.0% w/v to about 5.0% w/v.

The pharmaceutical composition of the present invention comprises Loteprednol etabonate in the concentration range from about 0.01% w/v to about 5% w/v; from about 0.05% w/v to about 2% w/v; from about 0.1% w/v to about 0.5% w/v.

The pharmaceutical composition of the present invention comprises lifitegrast and Loteprednol etabonate can be used in a different weight ratio.

The pharmaceutical composition according to the present invention further comprises one or more pharmaceutically acceptable excipients such as buffering agents, pH-adjusting agents, preservatives, osmotic/tonicity adjusting agents, viscosity modifying agents, antioxidants, surfactants or surface active agents, stabilizing agents, chelating agents, co-solvents, emollients, humectants and/or lubricants, vehicle, emulsifier or emulsifying agents and the like.

In another embodiment, the suitable buffering agents used in the present invention composition may include but not limited to any weak conjugate acid-base pair suitable for maintaining a desirable pH range. Useful examples include, but are not limited to, bicarbonate buffer, acetic acid, acetate buffer such as sodium acetate, citrate buffer such as sodium citrate, citric acid, lactic acid, sodium lactate, tartaric acid, sodium tartrate, tartrates, succinates, phosphate buffers such as sodium phosphate monobasic, sodium phosphate dibasic, phosphoric acid, borate buffer, or amino acids such as L-arginine, tromethamine (TRIS, 2-amino-2-hydroxymethyl-1,3-propanediol) buffer and its esters, hydrates, derivatives, analogues, side chains thereof and any combinations or mixtures thereof.

The pharmaceutical composition according to the present invention comprises buffering agents and their quantities will be selected so as to regulate the pH in the therapeutically useful range of pH 4-10, between 5-8.

In another embodiment, the suitable pH adjusting agents used in the present invention pharmaceutical composition may include but not limited to acids and bases. Suitable acids to adjust the pH of the composition include, but are not limited to, hydrochloric acid, glacial acetic acid, citric acid, sulfuric acid, nitric acid and/or mixture thereof. Suitable bases to adjust the pH of the composition include, but are not limited to, sodium hydroxide, potassium hydroxide, barium hydroxide, sodium acetate, sodium phosphate, amino acid and its esters, hydrates, derivatives, analogues, side chains thereof and any combinations or mixtures thereof.

In another embodiment, the suitable preservative used in the present invention pharmaceutical composition may include but not limited to quaternary ammonium salts such as benzalkonium chloride (BKC), benzalkonium bromide (BKB), benzododecinium bromide (BDD), benzethonium chloride, cetrimonium chloride, methyl parahydroxybenzoate, polyquaternium ammonium chloride, polyquaternium; alcohols, such as benzyl alcohol, chlorobutanol, phenylethanol and the like; phenol and its derivatives such as m-cresol and chlorocresol; mercury preservatives such as thimerosal, phenylmercuric acetate and phenylmercuric nitrate; guanidine-based preservatives including polyhexamethylene biguanide (PHMB), chlorhexidine and the like; sorbic acid and ophthalmically acceptable salts such sorbic acid, potassium sorbate; boric acid, borax, sodium perborate NaBO₃, and mixtures; salicylic acid, benzoic acid, lactic acid, acetic acid; oxidizing preservatives such as stabilized oxychloro complexes (e.g. Purite®), S.C.P (stabilized chlorite peroxide). Purite® is a registered trademark of Allergan, Inc., polyaminopropyl, and hydrogen peroxide, parabens such as methyl paraben, propyl paraben, ethyl paraben, butyl paraben; metal chlorites, such as alkali metal and alkaline earth metal chlorites and the like; per-salts such as per-borates, per-carbonates and its esters, hydrates, derivatives, analogues, side chains thereof and any combinations or mixtures thereof. The preservative may vary, and may include any compound or substance suitable for preventing microbial contamination in an ophthalmic formulation. The preservative is present in an amount from 0.005% w/v to 0.5% w/v of the composition.

In one embodiment, the present invention provides pharmaceutical compositions may contain an ionic or nonionic tonicity agent. The amount of tonicity agent will depend on the desired tonicity for the final formulation, but will generally be an amount sufficient to cause the formulations to have an osmolality of about 250-400 mOsm, the composition preferably has an osmolality of about 250-340 mOsm.

In another embodiment, the suitable osmotic/tonicity adjusting agents used in the present invention pharmaceutical composition may include but not limited to ionic and/or non-ionic type. Examples of ionic tonicity enhancers are alkali metal or earth metal halides, such as, for example, one or more of the following: calcium chloride, potassium chloride, sodium chloride, lithium chloride, potassium bromide, sodium bromide, sodium iodide, sodium phosphate, potassium phosphate, sodium and potassium sulfates, sodium and potassium bicarbonates, and boric acid. Non-ionic tonicity enhancing agents are, for example, urea, glycerin, glycerol, sorbitol, mannitol, sucrose, mannose, propylene glycol, dextrose and its esters, hydrates, derivatives, analogues, side chains thereof and any combinations or mixtures thereof. The amount of tonicity agent may vary depending upon whether an isotonic, hypertonic, or hypotonic liquid is desired. The tonicity adjusting agent is used in an amount to maintain the solution's osmolality compatible with respect to eye fluid. The tonicity adjusting agent may be used in concentrations ranging from about 0.05% w/v to about 0.51% w/v of the composition.

In another embodiment, the suitable viscosity modifying agents used in the present invention pharmaceutical composition may include but not limited to hydroxypropyl methylcellulose (HPMC), hydroxypropylcellulose (HPC), carboxymethylcellulose (CMC), methylcellulose (MC), hydroxyethylcellulose (HEC), cellulose and derivatives thereof, polycarbophil, polyoxyethylene glycol (PEG), hyaluronic acid (HA), amylase and derivatives thereof, amylopectins and derivatives thereof, dextran and derivatives thereof, polyvinylpyrrolidone (PVP), povidone, polyvinyl alcohol (PVA), and acrylic polymers such as derivatives of polyacrylic or polymethacrylic acid including hydroxylmethyl methacrylate (HEMA), carbomer such as carbomer 974 P, gums and/or resins include but not limited to guar gum; hydroxypropyl guar gum, gum arabic; karaya gum; xanthan gum; agar-agar; alginic acid, pullulan, chitosan, polysaccharide from tamarind seed, high-methoxyl pectin, low-methoxyl pectin, arabinogalactan, gellan gum, dextran, pemulen having two grades, pemulen TR-1 which holds up to 20% “oil” in an emulsion, and pemulen TR-2 which holds up to 50%), pemulen (high molecular weight copolymers of acrylic acid and C10-C30 alkyl acrylate crosslinked with allyl pentaerythritol), pemulen TR-2 (acrylates/C10-30 alkyl acrylate crosspolymer or high molecular weight, crosslinked copolymer of acrylic acid and a hydrophobic C10-30 alkyl acrylate co-monomer) and its esters, hydrates, derivatives, analogues, side chains thereof and any combinations or mixtures thereof.

In another embodiment, the suitable antioxidants used in the present invention pharmaceutical composition may include but not limited to sodium thiosulfate, sodium thiosulfate pentahydrate, sodium bisulfite, acetone sodium bisulfite, gentisic acid, gentisic acid ethanolamide, sodium formaldehyde sulfoxylate, thiourea, butylated hydroxyanisole (BHA), butylated hydroxy toluene (BHT), esters of gallic acid, ascorbic acid, salts of ascorbic acids such as ascorbyl palmitate, sodium ascorbate, retinoids and derivatives of vitamin A, acetylcysteine, thioglycerol, vitamin E and its derivatives e.g. vitamin E TPGS and its esters, hydrates, derivatives, analogues, side chains thereof and any combinations or mixtures thereof. Antioxidant may be used in concentrations ranging from about 0.05% w/v to about 1.0% w/v of the composition.

In another embodiment, the suitable surfactants or surface active agents or stabilizing agents used in the present invention pharmaceutical composition may include but not limited to, hydrophilic surfactants, lipophilic surfactants, amphiphilic surfactants and mixtures thereof. The examples of surface-active agents such as oleic acid, lecithin, sorbitan trioleate, polysorbate, benzododecinium bromide, cetylpyridinium chloride, tyloxapol, polyethylene glycol (PEG) glyceryl fatty acid esters, polyoxyethylene sorbitan monolaurate, polyoxyethylene sorbitan monostearate, polyoxyethylene sorbitan mono-oleate, octoxynol, polyoxyethylene (40) octyl phenyl ether (octoxynol 40), polyoxyethylene (40) isooctyl phenyl ether, polyoxypropylene/polyoxyethylene block copolymers, polyoxypropylene/polyoxyethylene/ethylene diamine block copolymers, ethoxylated castor oil; propylene glycol mono- and di-fatty acid esters, such as propylene glycol laurate, propylene glycol caprylate/caprate; and its esters, hydrates, derivatives, analogues, side chains thereof and any combinations or mixtures thereof. The surfactants or surface active agents or stabilizing agent present in the composition in concentrations ranging from about 0.001% w/v to about 1.0% w/v of the composition.

In another embodiment, the suitable chelating agents used in the present invention pharmaceutical composition may include but not limited to, edetate disodium, ethylenediamine tetracetic acid (EDTA), edetic acid, disodium edetate dihydrate, diethylenetriamine pentaacetic acid, nitrilotriacetic acid, hydraxyethylethylenediaminetriacetic acid, 1,2-diaminocyclohexane tetraacetic acid, hydroxyethylaminodiacetic polyphosphates, citric acid and its salts, tartaric acid and its salts, and its esters, hydrates, derivatives, analogues, side chains thereof and any combinations or mixtures thereof. The chelating agent present in the composition in concentrations ranging from about 0.001% w/v to about 0.5% w/v of the composition.

In another embodiment, the suitable co-solvents used in the present invention pharmaceutical composition may include but not limited to, propylene glycol, polyethylene glycol, glycerin, glycerol and its esters, hydrates, derivatives, analogues, side chains thereof and any combinations or mixtures thereof.

In another embodiment, the suitable vehicles/diluents used in the present invention pharmaceutical composition may include but not limited to water for injection, purified water, sterile water for injection (WFI), Ringer's solution, normal saline solution.

In another embodiment, the suitable oil phase used in the present invention pharmaceutical composition may include but not limited to, one or more of castor oil, polyglycerol oleate, propylene glycol monocaprylate, propylene glycol monolaurate, propylene glycol dicaprate and its esters, hydrates, derivatives, analogues, side chains thereof and any combinations or mixtures thereof.

In another embodiment, the suitable emulsifier/emulsifying agents used in the present invention pharmaceutical composition may include but not limited to, one or more of tween 80, castor oil, polyoxyethylene castor oil, polyoxyethylene hydrogenated castor oil, caprylic capric polyethylene glycol glyceride, lecithin, sodium lauryl sulfate (SLS), sodium dioctyl sulfosuccinate, tragacanth, spans, pemulen having two grades, pemulen TR-1 which holds up to 20% “oil” in an emulsion, and pemulen TR-2 which holds up to 50%), pemulen (high molecular weight copolymers of acrylic acid and C10-C30 alkyl acrylate crosslinked with allyl pentaerythritol), pemulen TR-2 (acrylates/C10-30 alkyl acrylate crosspolymer or high molecular weight crosslinked copolymer of acrylic acid and a hydrophobic C10-30 alkyl acrylate co-monomer) and its esters, hydrates, derivatives, analogues, side chains thereof and any combinations or mixtures thereof.

In another embodiment, the present invention provides a pharmaceutical composition, wherein the composition optionally may comprise preservatives.

In another embodiment, the present invention provides a pharmaceutical composition, wherein the composition is free of preservatives.

In another embodiment, the present invention pharmaceutical composition comprises one or more excipients and may vary depending on its dosage form and the route of administration is appropriately selected in accordance with the dosage form.

In another embodiment, the present invention pharmaceutical composition may varies depending on its dosage form and the route of administration is appropriately selected in accordance with the dosage form.

In one embodiment, the present invention pharmaceutical composition may be in the form of any suitable ophthalmic dosage form includes solution, suspension, emulsion, dispersion, ointment, gel or gel forming solution, nanoformulations, micellar solution, nanoemulsion, nanosuspension, ocular insert, implants, punctal plugs, microneedle, contact lenses, intraocular injection and the like.

In one embodiment, the present invention pharmaceutical composition is in the form solution, suspension, nano micellar emulsion, and the like.

In another embodiment, the present invention pharmaceutical composition may be suitable for sustained release or slow release formulations or extended release.

In one embodiment, the present invention provides pharmaceutical composition, wherein the composition can be packaged in various package forms known in the field of ophthalmic. In one embodiment, composition is filled into a sterile single-dose or multidose container; wherein the suitable containers include, but not limited to, bottles, vials or ampoules, single-use packs or vials or containers (i.e., the unit dose vials or containers), multidose preservative free container.

In another embodiment, the present invention provides pharmaceutical composition comprising combination of lifitegrast or pharmaceutically acceptable salts thereof and loteprednol or pharmaceutically acceptable salts thereof along with one or more pharmaceutically acceptable excipients, wherein said composition is filled into a single-dose/unit container.

In another embodiment, the present invention provides pharmaceutical composition comprising combination of lifitegrast or pharmaceutically acceptable salts thereof and loteprednol or pharmaceutically acceptable salts thereof along with one or more pharmaceutically acceptable excipients, wherein said composition is filled into a multi-dose container.

In another embodiment, the present invention provides pharmaceutical composition comprising combination of lifitegrast or pharmaceutically acceptable salts thereof and loteprednol or pharmaceutically acceptable salts thereof along with one or more pharmaceutically acceptable excipients, wherein said composition is filled into preservative free multi-dose container.

Pharmaceutically acceptable packaging materials include, but are not limited to, low density polyethylene (LDPE), linear low density polyethylene (LLDPE), high density polyethylene (HDPE), polypropylene (PP), polystyrene, polycarbonate, polyesters (such as polyethylene terephthalate and polyethylene naphthalate), nylon, polyvinyl chloride, poly(vinylidine chloride), poly(tetrafluoroethylene) and other materials known in the literature.

In one embodiment, the present invention pharmaceutical composition can be sterilized using any of the known methods of sterilization, such as filtration, moist heat, dry heat, gas sterilization or irradiation (gamma and electron beam).

In one embodiment, the present invention provides a pharmaceutical composition comprising: a) Lifitegrast or pharmaceutically acceptable salts thereof; b) Loteprednol or pharmaceutically acceptable salts thereof; and c) preservative; d) buffering agent; e) stabilizer and/or chelating agent; f) antioxidant; g) suspending agent; h) viscosity modifying agent; i) tonicity adjusting agent; j) vehicle;

In one embodiment, the present invention provides a pharmaceutical composition comprising: a) Lifitegrast; b) Loteprednol etabonate; c) benzalkonium chloride; d) sodium thiosulfate pentahydrate; e) EDTA; f) glycerin; g) povidone K-30; h) tyloxapol; i) sodium hydroxide and/or hydrochloric acid; j) sodium chloride; k) water for Injection;

In another embodiment, the present invention provides suspension composition comprises: a) Lifitegrast or pharmaceutically acceptable salts thereof; b) Loteprednol or pharmaceutically acceptable salts thereof; and c) preservative; d) buffering agent; e) stabilizer and/or chelating agent; f) suspending agent; g) surface active agents h) viscosity modifying agent; i) tonicity adjusting agent; j) vehicle and the like.

In one embodiment, the present invention provides suspension composition comprises: a) Loteprednol etabonate; b) Lifitegrast; c) benzalkonium chloride; d) glycerin; e) sodium citrate dihydrate; f) polyoxyethylene (40) octyl phenyl ether; g) sodium chloride; h) citric acid monohydrate; i) edetate disodium dihydrate; j) sodium hydroxide; k) water for injection and the like.

In one embodiment, the present invention provides suspension composition comprises: a) Loteprednol etabonate; b) Lifitegrast; c) benzalkonium chloride; d) glycerin; e) polyoxyethylene (40) octyl phenyl ether; f) sodium chloride; g) sodium phosphate dibasic anhydrous; h) edetate disodium dihydrate; i) sodium hydroxide; j) water for injection and the like.

In one embodiment, the present invention provides suspension composition comprises: a) Loteprednol etabonate; b) Lifitegrast; c) benzalkonium chloride; d) glycerin; e) sodium citrate dihydrate f) polyoxyethylene (40) octyl phenyl ether; g) xanthan gum; h) citric acid monohydrate; i) edetate disodium dihydrate; j) sodium hydroxide; k) water for injection and the like.

In one embodiment, the present invention provides suspension composition comprises: a) Loteprednol etabonate; b) Lifitegrast; c) benzalkonium chloride; d) glycerin; e) polyoxyethylene (40) octyl phenyl ether; f) xanthan gum; g) sodium phosphate dibasic anhydrous; h) edetate disodium dihydrate; i) sodium hydroxide; j) water for injection and the like.

In one embodiment, the present invention provides suspension composition comprises: a) Loteprednol etabonate; b) Lifitegrast; c) benzalkonium chloride; d) glycerin; e) sodium citrate dihydrate; f) polyoxyethylene (40) octyl phenyl ether; g) guar gum; h) citric acid monohydrate; i) edetate disodium dihydrate; j) sodium hydroxide; k) water for injection and the like.

In one embodiment, the present invention provides suspension composition comprises: a) Loteprednol etabonate; b) Lifitegrast; c) benzalkonium chloride; d) glycerin; e) polyoxyethylene (40) octyl phenyl ether; f) guar gum; g) sodium phosphate dibasic anhydrous; h) edetate disodium dihydrate; i) sodium hydroxide; j) water for injection and the like.

In one embodiment, the present invention provides suspension composition comprises: a) Loteprednol etabonate; b) Lifitegrast; c) benzalkonium chloride; d) glycerin; e) sodium citrate dihydrate; f) acrylates/C10-30 alkyl acrylate crosspolymer; g) sodium chloride; h) citric acid monohydrate; i) edetate disodium dihydrate; j) sodium hydroxide; k) water for injection and the like.

In one embodiment, the present invention provides suspension composition comprises: a) Loteprednol etabonate; b) Lifitegrast; c) benzalkonium chloride; d) glycerin; e) acrylates/C10-30 alkyl acrylate crosspolymer; f) sodium chloride; g) sodium phosphate dibasic anhydrous; h) edetate disodium dihydrate; i) sodium hydroxide; j) water for injection and the like.

In another embodiment of the present invention provides suspension composition comprising combination of lifitegrast and loteprednol along with one or more pharmaceutically acceptable excipients, wherein the excipients and their concentration range include benzalkonium chloride about 0.02 to about 0.20 mg; glycerin about 0.5 to about 25 mg; sodium citrate dihydrate about 1 to about 12 mg; polyoxyethylene (40) octyl phenyl ether about 0.01 to about 4 mg; sodium chloride about 0.5 to about 10 mg; citric acid monohydrate about 0.03 to about 6 mg; edetate disodium dihydrate about 0.01 to about 0.12 mg; sodium phosphate dibasic anhydrous about 1 to about 15 mg; xanthan gum about 0.02 to about 8 mg; guar gum about 0.02 to about 8 mg; acrylates/C10-30 alkyl acrylate crosspolymer about 0.01 to about 5 mg and the like.

In another embodiment of the present invention provides suspension composition comprising combination of lifitegrast and loteprednol along with one or more pharmaceutically acceptable excipients, wherein the excipients and their concentration range include benzalkonium chloride about 0.05 to about 0.15 mg; glycerin about 5 to about 15 mg; sodium citrate dihydrate about 4 to about 6 mg; polyoxyethylene (40) octyl phenyl ether about 0.2 to about 1 mg; sodium chloride about 2 to about 6 mg; citric acid monohydrate about 0.5 to about 3 mg; edetate disodium dihydrate about 0.05 to about 0.6 mg; sodium phosphate dibasic anhydrous about 3 to about 8 mg; xanthan gum about 1 to about 3 mg; guar gum about 1 to about 3 mg; acrylates/C10-30 alkyl acrylate crosspolymer about 0.05 to about 2 mg and the like.

In one embodiment, the present invention provides nanoemulsion composition comprises: a) Loteprednol etabonate; b) Lifitegrast; c) benzalkonium chloride; d) polysorbate 80; e) castor oil; f) glycerin; g) sodium citrate dihydrate; h) sodium chloride; i) citric acid monohydrate; j) edetate disodium dihydrate; k) sodium hydroxide; I) water for injection and the like.

In another embodiment of the present invention relates to a process for preparing a pharmaceutical composition comprising: a) Lifitegrast or pharmaceutically acceptable salts thereof; b) Loteprednol or pharmaceutically acceptable salts thereof; and c) one or more pharmaceutically acceptable excipients, wherein said composition can be prepared by suitable process known in the art.

In another embodiment of the present invention relates to a process for preparing a pharmaceutical composition comprising: a) Lifitegrast or pharmaceutically acceptable salts thereof; b) Loteprednol or pharmaceutically acceptable salts thereof; and c) one or more pharmaceutically acceptable excipients, wherein said process comprising the steps of:
i. take a vehicle of intended batch size into a compounding vessel;
ii. add dispensed quantity of lifitegrast and one or more pharmaceutically acceptable excipients;
iii. stir the content until clear colorless solution is observed;
iv. filter the solution and collect the filtered solution into holding vessel;
v. add the dispensed quantity of Loteprednol etabonate under stirring to the holding vessel and stir for specified time;
vi. makeup the volume to using vehicle & stir for specified time.

In another embodiment of the present invention relates to a process for preparing a pharmaceutical composition comprising: a) Lifitegrast or pharmaceutically acceptable salts thereof; b) Loteprednol or pharmaceutically acceptable salts thereof; and c) one or more pharmaceutically acceptable excipients, wherein said process may varies depending on selection of dosage form of composition.

In one embodiment, the present invention provides a method for treatment of dry eye disease, wherein the method comprises administering a pharmaceutical composition comprising: a) Lifitegrast or pharmaceutically acceptable salts thereof; b) Loteprednol or pharmaceutically acceptable salts thereof; and c) one or more pharmaceutically acceptable excipients. In another embodiment, the present invention provides a method for treatment of dry eye disease, wherein the method comprises administering a pharmaceutical composition comprising: a) Lifitegrast; b) Loteprednol etabonate; and c) one or more pharmaceutically acceptable excipients.

In one embodiment, the present invention provides pharmaceutical composition comprising: a) Lifitegrast or pharmaceutically acceptable salts thereof; b) Loteprednol or pharmaceutically acceptable salts thereof; and c) one or more pharmaceutically acceptable excipients, wherein the said composition is administered topically to eye of subject for reducing signs and symptoms of dry eye disease.

In another embodiment, the present invention provides pharmaceutical composition comprising: a) Lifitegrast; b) Loteprednol etabonate; and c) one or more pharmaceutically acceptable excipients, wherein the said composition is administered topically to eye of subject for reducing signs and symptoms of dry eye disease.

In one embodiment, present invention provides pharmaceutical composition is topically administered to the eyes once daily, two times daily, three times daily, four times daily.

In another embodiment, present invention provides pharmaceutical composition is administered daily, weekly, or monthly based upon the dosages, dosage form and route of administration.

The dosage and dosage regimen may be varied based on patient, treatment condition, severity of condition being treated, the route of administration, etc. to achieve desired therapeutic effect.

The following Examples illustrate numerous compositions of the present invention. These formulations are merely illustrative and not limitative of the remainder of the specification and claims in any way whatsoever.

The present invention is further exemplified with following examples and is not intended to limit the scope of the invention.

### EXAMPLES

### Example 1: An Exemplary Suspension Composition as Follows

| | | Quantity (mg) | | | | | |
|---|---|---|---|---|---|---|---|
| SI. | Ingredients | F-I | F-II | F-III | F-IV | F-V | F-VI |
| 1 | Loteprednol | 2.5 | 2.5 | 2.5 | 2.5 | 2.5 | 2.5 |
| | etabonate | | | | | | |
| 2 | Lifitegrast | 50 | 50 | 50 | 50 | 50 | 50 |
| 3 | Benzalkonium | 0.10 | 0.10 | 0.10 | 0.10 | 0.10 | 0.10 |
| | chloride | | | | | | |
| 4 | Glycerin | 6.09 | 6.09 | 13 | 13 | 13 | 13 |
| 5 | Sodium citrate | 5.16 | — | 5.16 | — | 5.16 | — |
| | dihydrate | | | | | | |
| 6 | polyoxyethylene | 0.5 | 0.5 | 0.5 | 0.5 | 0.5 | 0.5 |
| | (40) octyl phenyl | | | | | | |
| | ether | | | | | | |
| 7 | Xanthan gum | — | — | 1.2 | 1.2 | — | — |
| 8 | Guar gum | — | — | — | — | 1.2 | 1.2 |
| 9 | Sodium chloride | 2.1 | 2.1 | — | — | — | — |
| 10 | Citric acid | 0.67 | — | 0.67 | — | 0.67 | — |
| | monohydrate | | | | | | |
| 11 | Sodium phosphate | — | 3.67 | — | 3.67 | — | 3.67 |
| | dibasic anhydrous | | | | | | |
| 12 | Edetate disodium | 0.53 | 0.53 | 0.53 | 0.53 | 0.53 | 0.53 |
| | dihydrate | | | | | | |
| 13 | Sodium hydroxide | q.s to | q.s to | q.s to | q.s to | q.s to | q.s to |
| | | pH | pH | pH | pH | pH | pH |
| 14 | Water for injection | q.s to | q.s to | q.s to | q.s to | q.s to | q.s to |
| | | 1 ml | 1 ml | 1 ml | 1 ml | 1 ml | 1 ml |

pH 5.0-8.0, q.s—quantity sufficient.

### Manufacturing Procedure

### A). Preparation of Slurry Phase (20%) for Formulation I, II, III, IV, V, VI:

1. Initial 10% of water for injection (WFI) was taken.
2. Dispensed quantity of polyoxyethylene (40) octyl phenyl ether added to above water for injection (WFI).
3. Dispensed quantity of benzalkonium chloride added to above phase.
4. Dispensed quantity of loteprednol etabonate (API) added to above phase.
5. Above phase passed through nano-mill to get required particle size.
   B) Preparation of Buffer Phase (80%) for Formulation I, II and Buffer Phase (40%) for formulation III, IV, V, VI:
1. Initial 50% of water for injection (FI, FII), Initial 20% of water for injection (FIII, FIV, FV, FVI) was taken.
2. Dispensed quantity of edetate disodium dihydrate added to above WFI.
3. Dispensed quantity of glycerine added to above phase.
4. Dispensed quantity of sodium citrate dihydrate (FI, FIII, FV), sodium phosphate dibasic anhydrous (FII, FIV, FVI) added to above phase.
5. Dispensed quantity of sodium chloride (FI, FII) added to above phase.
6. Dispensed quantity of citric acid monohydrate (FI, FIII, FV) added to above phase.
7. Dispensed quantity of lifitegrast (API) added to above phase.
8. The pH of above phase adjusted to 7.5 by using 1N sodium hydroxide (NAOH).
9. The volume of above phase made up to require amount by using nitrogen purged WFI.

### C) Preparation of Polymer Phase (20%) for Formulation III, IV, V, VI:

1. Initial 10% of Water for injection (WFI) was taken in clean and dried Duran bottle.
2. Dispensed quantity of xanthan gum (FIII, FIV), guar gum (FV, FVI) added to above phase.
3. The volume of above phase made up to 20% by using nitrogen purged WFI.

### D) Preparation of Final Composition:

### Formulation I, II:

1. From initially prepared buffer phase, 80% buffer phase was taken in to clean and dried Duran bottle.
2. Then 20% milled slurry phase added to above phase.
3. Above phase allowed to stir for 30 min at 500 RPM to get uniform suspension.

### Formulation III, IV, V, VI:

1. From initially prepared buffer phase, 40% buffer phase was taken in clean and dried Duran bottle.
2. 20% milled slurry phase added in above phase and then added 20% polymer phase.
3. Volume of above phase made up to 100%.

We claim:

1. A pharmaceutical composition comprising:
a) Lifitegrast or pharmaceutically acceptable salts thereof;
b) Loteprednol or pharmaceutically acceptable salts thereof; and
c) one or more pharmaceutically acceptable excipients.

2. The pharmaceutical composition as claimed in claim 1; wherein said composition comprises Lifitegrast at a concentration range from about 1.0% w/v to about 5.0% w/v.

3. The pharmaceutical composition as claimed in claim 1; wherein composition comprises Loteprednol etabonate at a concentration range from about 0.1% w/v to about 0.5% w/v.

4. The pharmaceutical composition as claimed in claim 1; wherein one or more excipient comprises buffering agents, pH-adjusting agents, preservatives, osmotic/tonicity adjusting agents, viscosity modifying agents, antioxidants, surfactants or surface active agents, stabilizing agents, chelating agents, vehicle and the like.

5. The pharmaceutical composition as claimed in claim 4; wherein the buffering agents include sodium citrate dihydrate, sodium phosphate dibasic anhydrous, citric acid monohydrate; pH-adjusting agent include sodium hydroxide; preservatives include benzalkonium chloride; osmotic/tonicity adjusting agent include sodium chloride, gycerin; viscosity modifying agents include xanthan gum, guar gum; surfactants or surface active agents include polyoxyethylene (40) octyl phenyl ether; chelating agent include edetate disodium dihydrate; vehicle include water for injection and combinations thereof.

6. The pharmaceutical composition as claimed in claim 1; wherein the pharmaceutical composition having osmolality is about 250 to 400 mOsm.

7. The pharmaceutical composition as claimed in claim 1; wherein said composition is in the form of solution, suspension, emulsion, gel, gel forming solution, dispersion, nano-formulations, insert, implant, punctal plugs and the like.

8. The pharmaceutical composition as claimed in claim 7; wherein pharmaceutical composition in the form of suspension, solution, nano micellar emulsion.

9. The pharmaceutical composition as claimed in claim 1; wherein said composition used for the treatment of dry eye disease.

10. The pharmaceutical composition as claimed in claim 1; wherein said composition is administered topically to an eye of the subject.
